(19) United States
(12) Plant Patent Application Publication
(10) Pub. No.: US 2009/0178171 P1
(43) Pub. Date: Jul. 9, 2009

(54) Ageratum plant named 'Agbilir'

(50) ;

(75) Inventor: Monica Maria Adelheid Sanders, Grootebroek (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 12/006,528

(22) Filed: Jan. 3, 2008

(51) Int. Cl. A01H 5/00

(52) U.S. Cl. PLT/400

(57) ABSTRACT

A new Ageratum plant named ‘Agbilir’ particularly distinguished by its large, mid purple capitula with light purple stigma when opening, its early flowering, its strong branching and vigorous growth.

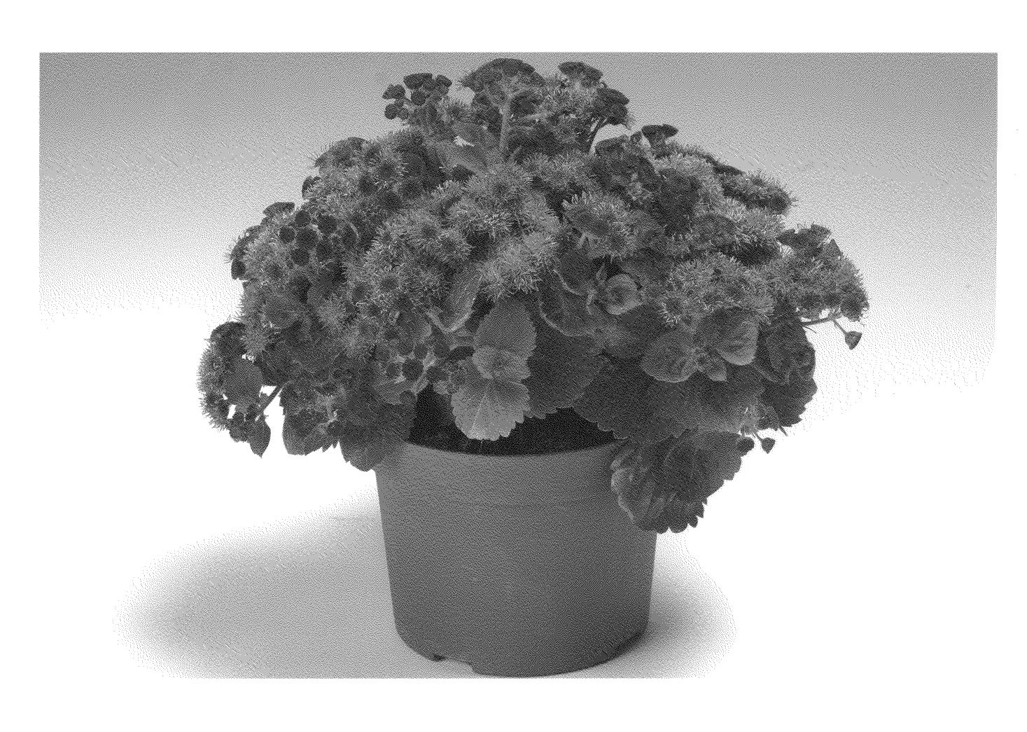

### LATIN NAME OF THE GENUS AND SPECIES OF THE PLANT CLAIMED

Ageratum houstonianum

### VARIETAL DENOMINATION

‘Agbilir’

### BACKGROUND OF THE NEW PLANT

The present invention comprises a new and distinct cultivar of Ageratum, botanically known as Ageratum houstonianum, and herinafter referred to by the cultivar name ‘Agbilir.’ The new cultivar is propagated from cuttings, resulting from a cross-pollination in September 2002 of a proprietary selection of Ageratum housionianum identified as number ‘X52-4,’ not patented, as the female, or seed parent, with a proprietary selection of Ageratum houstonianum identified as number ‘X101-7,’not patented, as the male, or pollen parent.

As a result the present cultivar was found in 2003 in Enkhuizen, Netherlands and has been repeatedly asexually reproduced by cuttings in Enkhuizen, Netherlands, in Gilroy, Calif., USA, in Angers, France and in Sarrians, France since 2003.

The distinctive characteristics of this new Ageratum are stable and reproduced true to type in successive generations of asexual reproduction. It takes 7 to 9 weeks to produce a finished plant, depending on the temperature.

This new Ageratum plant is an annual in most climatic zones in the US. It is a perennial plant only in zones 9 and 10.

### SUMMARY OF THE INVENTION

The following traits have been repeatedly observed and are determined to be the unique characteristics of ‘Agbilir.’ These characteristics in combination distinguish ‘Agbilir’ as a new and distinct Ageratum cultivar:
1. Compact, upright and mounded growth habit
2. Freely branching habit
3. Freely flowering habit with many large capitula in compound umbels
4. Mid purple capitula that turn light purple when flowers open
5. Medium sized, green coloured leaves

Plants of the new Ageratum differ primarily from plants of the female parent selection in the following characteristics:

Plants of the new Ageratum have a compact plant habit whereas plants of the female parent selection have a more vigorous plant habit.

Plants of the new Ageratum differ primarily from plants of the male parent selection in the following characteristics:

Plants of the new Ageratum have mid purple capitula with mid violet stigma whereas plants of the male parent selection have deep purple capitula.

TABLE I
DIFFERENCES BETWEEN THE NEW CULTIVAR
‘AGBILIR’ AND A SIMILAR CULTIVAR

| | | ‘AGROSAMTIS’ (U.S. Plant |
|---|---|---|
| | ‘AGBILIR’ | Pat. No. 15,631) |
| Color of capitulum at | RHS 72A | RHS 64D |
| beginning of flowering | | |
| Color of capitulum at full | RHS 72D | RHS 65A |
| flowering | | |
| Plant height | 25 cm | 15-20 cm |
| Spreading area of plant | 46 cm | 30-35 cm |

### DESCRIPTION OF THE NEW CULTIVAR

The following detailed descriptions set forth the distinctive characteristics of this new Ageratum. The data which define these characteristics were collected from asexual reproductions carried out in Enkhuizen, Netherlands. The plant history was taken on 30 week old plants, blossomed under natural light and artificial light in a greenhouse.

Color readings were taken in laboratory under ambient light. Color references are primarily to the RHS Colour Chart of The Royal Horticultural Society of London.
The plant:
   Classification.—botanical Ageratum houstonianum.
Parentage:
   Female parent.—Proprietary selection ‘X52-4’, not patented.
   Male parent.—Proprietary selection ‘X101-7’, not patented.
Plant description:
   Growth habit.—Upright.
   Branching habit.—Freely branching; lateral branches at every node.
   Plant height.—25 cm.
   Spreading area of plant.—46 cm.
   Vigour.—Strong.
   Strength.—Very strong.
   Blooming period.—Year round.
The stem:
   Length.—29 cm.
   Diameter.—3 mm.
   Shape of cross section.—Round.
   Color.—RHS 144A.
   Anthocyanin pigmentation.—Absent.
   Internode length.—25-30 mm, depending on light intensity.
   Pubescence.—Slightly pubescent.
The foliage:
   Phyllotaxis.—Opposite, decussate.
   Shape of blade.—Ovate.
   Texture upper and lower side.—Slightly pubescent.
   Venation.—Reticulate.
   Margin.—Serrate.
   Base.—Cordate.
   Apex.—Acute.
   Length.—30 mm.
   Width.—31 mm.
   Color upper side.—RHS 137A.
   Color lower side.—RHS 137C.
   Pubescence.—Slightly pubescent.
   Petiole.—Length: 7 mm Diameter: 2 mm Color RHS: 139C Texture: Slightly pubescent.
The inflorescence:
   Inflorescence type.—Compound umbel of capitula.
   Number of inflorescences per plant.—120-130.
   Umbel size.—4.2 cm.
   Umbel depth.—2.4 cm.
   Peduncle length.—11 mm.
   Peduncle diameter.—2 mm.
   Peduncle color.—RHS 144A.
   Pedicel length.—0.9 mm.
   Pedicel diameter.—1 mm.
   Pedicel color.—RHS 144B.
   Number of capitula per inflorescence.—21.
   Number of disc florets per capitulum.—Approximately 85.
   Number of ray florets per capitulum.—0.
   Capitulum shape in bud stage.—Round, flat, showing unopened florets.
   Disk florets.—Corolla shape: Actinomorphic Number of lobes: 5 Length: 0.8 mm Diameter: 1 mm Color upper side: RHS 63B Color lower side RHS N155B.
   Phyllaries.—Number of phyllaries per capitulum: 20 Length: 2 mm Diameter 0.2 mm Color outer side: RHS 138A with tip RHS 185A Color inner side: RHS 138C with tip RHS 185B.
   Capitulum.—Diameter at begin of flowering: 5 mm Diameter at full flowering: 6 mm Depth 6 mm Color at begin of flowering: RHS 72A Color at full flowering: RHS 72D.
   Lastingness of the bloom.—The capitula of one umbel open over a period of 3 to 4 weeks.
Reproductive organs:
   Number of pistils.—1.
   Pistil shape.—Style with two filiform branches.
   Pistil length.—6 mm.
   Stigma color.—RHS 75B.
   Ovary type.—Inferior; 5-ribbed.
   Pappus.—Coroniform.
   Number of anthers.—5, connate in a tube, filaments free.
   Shape of anthers.—Ecalcarate.
   Pollen amount.—Small.
   Pollen color.—RHS 155B.
   Fragrance.—Absent.
Seed development: Seed development has not been observed to date.
Roots:
   Type of roots.—Fibrous. Roots start to grow on every part of the stem that contacts the soil, so not only at the nodes.
Disease/pest resistance: No disease/pest resistance has been observed to date.
Temperature tolerance: Plants of the new Ageratum have been observed to be tolerant to temperatures ranging from 4 to 30° C.

What is claimed is:

1. A new and distinct variety of Ageratum plant named ‘Agbilir,’ as substantially illustrated and described herein.
